(19) United States
(12) Patent Application Publication
(10) Pub. No.: US 2020/0368120 A1
(43) Pub. Date: Nov. 26, 2020

(54) POROUS COMPOSITE PARTICLES AND COSMETIC COMPOSITIONS COMPRISING THE SAME

(71) Applicant: Conopco, Inc., d/b/a UNILEVER, Trumbull, CT (US)

(72) Inventors: Guanghui MA, Beijing (CN); Xiangming NA, Beijing (CN); Amitava PRAMANIK, Bangalore (IN); Weiqing ZHOU, Beijing (CN); Weizheng ZHOU, Shanghai (CN)

(73) Assignee: Conopco, Inc., d/b/a UNILEVER, Trumbull, CT (US)

(21) Appl. No.: 16/769,702

(22) PCT Filed: Nov. 30, 2018
(86) PCT No.: PCT/EP2018/083113

§ 371 Date: Jun. 4, 2020

(30) Foreign Application Priority Data
Dec. 22, 2017 (CN) PCT/CN2017/118006
Jan. 23, 2018 (EP) 18153047.8

(51) Int. Cl. A61K 8/06; A61K 8/27; A61K 8/28; A61K 8/29; A61K 8/81; A61Q 19/02; A61Q 1/02

(52) U.S. Cl. CPC A61K 8/066; A61K 8/27; A61K 8/28; A61K 8/29; A61K 8/8147; A61K 2800/654; A61Q 19/02; A61Q 1/02; A61K 2800/412; A61K 2800/43; A61K 2800/651; A61K 8/8152

(57) ABSTRACT

Disclosed are porous composite particles and compositions comprising the same. The porous composite particles comprising base of an amphiphilic or hydrophobic polymer and 0.1 to 20% of a pigment based on the total weight of porous composite particles, where said particles have 50 to 95 vol % internal porosity and an outer surface which is either continuous and devoid of pores or is discontinuous comprising plurality of pores whose total area is 0.01 to 30% of the total outer surface area. When typically applied, the compositions impart improving the characteristics of skin.

### FIELD OF THE INVENTION

The present invention relates to porous composite particles as well as a cosmetic composition comprising porous composite particles.

### BACKGROUND OF THE INVENTION

Many consumers desire to look radiant and natural instead of getting an artificial matte look by using traditional foundation-based products, which tend to be overly opaque in nature and may have aesthetic and/or cultural negatives. It is the current trend that more and more make-up products are promoted to have moisturizer type in-use sensory and traditional moisturizers are formulated to deliver more perceivable optical benefits.

There has been considerable effort by the cosmetics industry to provide compositions which can enhance the skin appearance benefits, such as lightening/whitening, natural looking skin and even-toned skin. Often whitening/lightening is achieved by using materials such as particles with high refractive indices such as micronized titanium dioxide and zinc oxide and other inorganic particulates. An even skin tone or pinkish skin tone is often delivered by particulate pigments and colorants. However, it is difficult to deliver desired skin appearance by only using conventional pigments or colorants owing to the nature of those materials. Further, high loading of particles can lead to poor tactile sensories and for this reason moisturizers typically maintain low pigment dosage. Further, it is not very easy to formulate high loading of conventional pigments or colorants into moisturizers.

There is an increasing interest to develop composite particles with better compatibility with other usual cosmetically acceptable ingredients and to develop compositions with such composite particles that yield desired skin appearance, such as lightening/whitening, natural looking skin and even-toned skin.

U.S. Pat. No. 9,469,738 B (Eastman Kodak, 2016) discloses a foamable aqueous composition comprises porous polymeric particles, a binder material, water, an opacifying colorant and various additives, and has a foam density of at least 0.1 g/cm³ to 0.5 g/cm³. The foamable aqueous composition can be aerated, disposed on a porous substrate, dried, and crushed on that porous substrate. The composition can be used to form light-blocking and light-colored foamed, opacifying elements with one or more dry foamed layers.

US2015234098 A (Eastman Kodak) discloses a light blocking article containing porous particles comprising a continuous polymeric binder and pores within the continuous polymeric binder, an opacifying colorant that absorbs the predetermined electromagnetic radiation, and a matrix polymer in which the porous particles and opacifying colorant are dispersed. The article is designed to be lightweight but effective to block most incident actinic radiation and can be designed into fabrics, curtains, and other materials. Such an article has an opacifying layer that is capable of blocking predetermined electromagnetic radiation.

US2016317401 A (L'Oreal) discloses a composite particle comprising a matrix containing at least one inorganic and/or at least one organic material and at least one inorganic UV filter which is doped by at least one transition metal. It also discloses a composition containing, in a physiologically acceptable medium, at least the composite particles as defined above. The composition is more particularly intended for the photoprotection of the skin and/or hair against ultraviolet (UV) radiation.

JP2005350430 A (Mitsubishi Pencil Co.) discloses a cosmetic comprises a composite porous product containing a water soluble organic polymer in a metal oxide. The manufacturing method therefor comprises incorporating a porous product synthesized by mixing the water soluble organic polymer with a metal oxide precursor, causing the phase separation between the metal oxide precursor and the water soluble organic polymer, and subjecting the metal oxide precursor to ceramicization as a cosmetic.

US2013287826 A (Unilever) discloses composite particles and compositions comprising the composite particles. The particles have a polymeric base with or without voids and some subparticles, whereby the subparticles have indices of refraction above and below the index of refraction of the polymeric base. When typically applied, the compositions impart excellent soft focus results on the skin of consumers.

CN103374143 A (Inst Process Eng CAS) discloses super macroporous polymer microspheres and a preparation method thereof. The super macroporous polymer microspheres can be used as stationary phase fillers for chromatographic separation, immobilized carriers of enzymes, cell culture micro-carriers, tissue engineering micro scaffold materials and adsorbing materials.

EP1964875 A1 (Ube Industries Ltd.) discloses powder comprising fine inorganic compound particles-deposited porous polyamide particles in which the fine inorganic compound particles are deposited on surfaces and in pores of the porous polyamide particles, and a cosmetic composition comprising the powder. The composition is not harmful to human body and inhibits abnormal shining on skin and shields defective skin conditions.

Usually the composite particles are prepared by using conventional templates which result in low internal porosity (less than about 50 vol %) or irregular air pockets which are responsible for the internal porosity. When used in cosmetic compositions, such particles are unable to meet the high standards of opacity on the skin which are demanded by consumers or users of such cosmetic compositions.

### SUMMARY OF THE INVENTION

The present inventors have developed novel porous composite particles with high porosity and cosmetic compositions comprising such particles for skin characteristics such as natural looking, even-toned and translucent pinkish white skin. Usually such composite particles need a lot of energy to be uniformly mixed or dispersed in the matrix or carrier of the concerned cosmetic compositions such as creams. However, we have determined that porous composite particles of the invention could be mixed or dispersed uniformly in a carrier using significantly lesser energy, thereby improving the sustainability factor of the process.

In accordance with a first aspect is disclosed porous composite particles comprising base of an amphiphilic or hydrophobic polymer and 0.1 to 20% of a pigment based on the total weight of porous composite particles, where said particles have 50 to 95 vol % internal porosity and an outer surface which is either continuous and devoid of pores or is discontinuous comprising plurality of pores whose total area is 0.01 to 30% of the total outer surface area.

In a second aspect is disclosed a process to prepare the porous composite particle of the first aspect of this invention, comprising the steps of:
(i) forming a w/o emulsion comprising a water phase and an oil phase, said oil phase comprising said polymer, an organic solvent for solubilizing the polymer and said pigment;
(ii) further emulsifying said w/o emulsion in an outer water phase comprising an emulsifier to form a w/o/w double emulsion;
(iii) mixing said w/o/w double emulsion with additional water to allow said solvent to diffuse into said additional water to precipitate said porous composite particles.

In a third aspect is disclosed a cosmetic composition comprising the porous composite particles of the first aspect of this invention.

In a fourth aspect is disclosed a method of improving characteristics of skin by applying to skin a cosmetic composition comprising the porous composite particles of the first aspect of this invention.

In a fifth aspect is disclosed use of a cosmetic composition comprising the porous composite particles of the first aspect of this invention for improving skin characteristics.

All other aspects of the present invention will more readily become apparent upon considering the detailed description and examples which follow.

Except in the examples, or where otherwise explicitly indicated, all numbers in this description indicating amounts of material or conditions of reaction, physical properties of materials and/or use may optionally be understood as modified by the word “about”.

All amounts are by weight of the cosmetic composition, unless otherwise specified.

It should be noted that in specifying any ranges of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

### DETAILED DESCRIPTION

By “a cosmetic composition” as used herein, is meant to include a composition for topical application to the skin of mammals, especially human beings. Such a composition may be generally classified as leave-on or rinse off but is preferably of the leave on type. The composition is formulated into a product which is applied to a human body specifically for improving appearance but may, in addition, also provide cleansing, odor control or general aesthetics. The composition of the present invention can be in the form of a liquid, lotion, cream, foam, scrub, gel, or toner, or applied with an implement or via a face mask or a pad. Non-limiting examples of such compositions include leave-on skin lotions, creams, antiperspirants, deodorants, lipsticks, foundations, mascara, sunless tanners and sunscreen lotions. The composition of the present invention is preferably a leave-on composition. “Skin” as used herein is meant to include skin on the face and body (e.g., neck, chest, back, arms, underarms, hands, legs, buttocks and scalp) and especially to the sun exposed parts thereof.

“Porous composite particles” as used herein refers to particles with plurality of distributed air pockets throughout the entire volume of the particles. The air pockets could be disconnected or inter-connected by small size openings, similar to pore openings that separate larger spaces.

### Internal Porosity

The term “internal porosity” as used herein refers to the porosity which is entirely due to air pockets within the porous composite particles. The internal porosity can be measured by using Mercury intrusion porosimetry. It is calculated by the volume of voids over the total volume of the particles and is expressed as a percentage between 0 vol % and 100 vol %.

The internal porosity of the porous composite particles in accordance with this invention is 50 to 95 vol %, more preferably 65 to 95 vol %, most preferably 70 to 95 vol %.

Porous composite particles in accordance with this invention preferably comprise plurality of air pockets which can either be interconnected or disconnected. Preferably, the internal porosity is due to plurality of disconnected air pockets. Alternatively, the internal porosity is due to plurality of interconnected air pockets.

### Outer Surface

Porous composite particles in accordance with this invention comprise an outer surface which is either continuous and devoid of pores or is discontinuous comprising plurality of pores whose total area is 0.01 to 30% of the total outer surface area. It is preferred that the outer surface is discontinuous comprising plurality of pores. Preferably, total area of the plurality of pores is 0.1 to 10% of said total outer surface area.

### Shape of Particles

Porous composite particles in accordance with this invention could be of any shape, regular or irregular. However, the composite particles of the invention are preferably spherical or spheroidal.

It is particularly preferred that the diameter of the porous composite particles is 0.5 to 100 microns, and more preferably 1 to 50 microns, and most preferably 3 to 10 microns.

### Pigment

Porous composite particles in accordance with this invention comprise 0.1 to 20% of a pigment, preferably 0.1 to 10% of a pigment, and more preferably 0.5 to 5% of a pigment. Preferably, the particle size of such pigment is 0.01 to 5 microns, and more preferably 0.05 to 1 micron. When the particles are spherical or spheroidal, the particle size is the diameter of such particles.

The pigment is preferably inorganic. Alternatively, the pigment is organic. Preferably the inorganic pigment is zinc oxide, titanium dioxide, zirconium oxide, iron oxide or a mixture thereof.

it is usually found that skin whitening caused by a white pigment can occasionally appear in uneven “patches” even when the cosmetic composition comprising the pigment is applied evenly. Presence of a non-white pigment can ameliorate this effect. Thus, it is preferred that the composition of this invention comprises a combination of pigments comprising a white pigment and non-white pigment. In such a situation, more preferably, a majority of said combination is composed of non-white pigment.

It is particularly preferred that the porous composite particles of the invention comprise a combination of pigments comprising a white pigment and non-white pigment, wherein said white pigment is zinc oxide and said non-white pigment is iron oxide. Alternatively, said white pigment is titanium oxide and said non-white pigment is iron oxide.

### The Polymeric Base

Porous composite particles in accordance with this invention comprise a polymeric base. The limitation with respect to the polymer base that may be used in this invention is that the polymeric base is a base of an amphiphilic polymer or hydrophobic polymer.

Amphiphilic polymer is a polymer composed of hydrophilic (water-loving) and hydrophobic (water-repellent) parts.

Hydrophobic polymer is a polymer which repels water, which means lacking affinity for water, and tending to repel or not to absorb water.

It is preferred that the amphiphilic polymer is selected from methoxy polyethylene glycol polylactic acid copolymer, polyoxyethylene and polyoxypropylene copolymer, polystyrene and polyethylene glycol copolymer, polystyrene and polyacrylate copolymer, polystyrene and polyacrylate hydroxyethyl ester copolymer, polymethyl methacrylate and polyacrylate copolymer, polymethyl methacrylate and polyacrylate hydroxyethyl ester copolymer, methyl methacrylate and methacrylate copolymer, methyl methacrylate, β-hydroxyethyl methacrylate copolymer or mixture thereof.

It is preferred that the hydrophobic polymer is selected from polystyrene, polychloromethylstyrene, polyglycidyl methacrylate or a mixture thereof.

### Double Emulsion Method

“Double emulsion” as used herein refers to water-in-oil-in-water type (W/O/W). The first step of a double emulsion method is the formation of a water in oil (w/o) emulsion where the aqueous solution and the oil phase contains hydrophobic active components and a suitable surfactant with low HLB. The miniemulsion is formed under strong shear stress (i.e. sonication, microfluidization, high speed homogenization). Next, the water in oil in water (w/o/w) double emulsion is sonicated or homogenized for droplet-size reduction. Evaporation, the final step, is used to remove the organic solvent.

Porous composite particles in accordance with this invention are preferably prepared by using emulsion droplets as template, and more preferably by using double emulsion method.

### Process for Preparation of Porous Composite Particles:

This invention is also directed to a process to prepare porous composite particles. The process comprises the steps of:
(i) forming a w/o emulsion comprising a water phase and an oil phase, said oil phase comprising said polymer, an organic solvent for solubilizing the polymer and said pigment;
(ii) further emulsifying said w/o emulsion in an outer water phase comprising an emulsifier to form a w/o/w emulsion;
(iii) mixing said w/o/w emulsion with additional water to allow said solvent to diffuse into said additional water to precipitate said porous composite particles.

Preferably, the water phase of step (i) comprises a salt. Preferably, the outer water phase of step (ii) comprises a salt.

It is preferred that the salt used in said step (i) and step (ii) is selected from sodium chloride, potassium chloride, magnesium chloride, sodium sulfate, potassium sulfate or magnesium sulfate. More preferably the same salt is used in either steps.

Preferably the organic solvent is selected from chloroform, acetone, tetrahydrofuran, dichloromethane, chloroform, dichloroethane or ethyl acetate. The nature of the solvent (e.g., polarity, dipole moment) needs to be considered while choosing the appropriate solvent for a given or chosen amphiphilic or hydrophobic polymer. Generally, the principles of like-dissolves-like are employed to select an appropriate solvent.

Preferably, the emulsifier is a nonionic emulsifier or an anionic emulsifier. Preferably the anionic emulsifier is sodium dodecyl sulfate (SDS).

### Cosmetic Compositions

In accordance with a further aspect is disclosed a cosmetic composition comprising porous composite particles of the first aspect in a cosmetically acceptable carrier. It is preferred that the cosmetic composition comprises from 0.1 to 20%, and more preferably, from 0.2 to 15%, and most preferably from 0.75 to 6% by weight porous composite particles, based on total weight of the composition.

A variety of materials may be present in the compositions comprising the composite particles of this invention to serve as cosmetically acceptable carriers. Preferably, the cosmetically acceptable carrier is an emulsion. In one aspect the carrier is a water-in-oil emulsion. Alternatively, the carrier is an oil-in-water emulsion.

Preferably the carrier comprises water. Amounts of water may, for example, range from 1 to 85 wt %, more preferably from 5 to 90%, even more preferably from 35 to 80%, optimally between 40 and 70% by weight of the cosmetic composition, depending on the nature of the composition.

Further preferably the compositions of the invention comprise a skin lightening agent. The skin lightening agent is preferably chosen from one or more of a vitamin B3 compound or its derivative e.g. niacin, nicotinic acid or niacinamide or other well-known skin lightening agents e.g. adapalene, aloe extract, ammonium lactate, anethole derivatives, apple extract, arbutin, azelaic acid, kojic acid, bamboo extract, bearberry extract, bletilla tuber, Bupleurum falcatum extract, burnet extract, butyl hydroxy anisole, butyl hydroxy toluene, citrate esters, Chuanxiong, Dang-Gui, deoxyarbutin, 1,3-diphenyl propane derivatives, 2,5-dihydroxybenzoic acid and its derivatives, 2-(4-acetoxyphenyl)-1,3-dithane, 2-(4-hydroxyphenyl)-1,3-dithane, ellagic acid, escinol, estragole derivatives, Fadeout (Pentapharm), Fangfeng, fennel extract, ganoderma extract, gaoben, Gatuline Whitening (Gattlefosse), genistic acid and its derivatives, glabridin and its derivatives, gluco pyranosyl-1-ascorbate, gluconic acid, glycolic acid, green tea extract, 4-hydroxy-5-methyl-3[2H]-furanone, hydroquinone, 4-hydroxyanisole and its derivatives, 4-hydroxy benzoic acid derivatives, hydroxycaprylic acid, inositol ascorbate, lemon extract, linoleic acid, magnesium ascorbyl phosphate, Melawhite (Pentapharm), Morus alba extract, mulberry root extract, 5-octanoyl salicylic acid, parsley extract, Phellinus linteus extract, pyrogallol derivatives, 2,4-resorcinol derivatives, 3,5-resorcinol derivatives, rose fruit extract, salicylic acid, Song-Yi extract, 3,4,5-trihydroxybenzyl derivatives, tranexamic acid, vitamins like vitamin B6, vitamin B12, vitamin C, vitamin A, dicarboxylic acids, resorcinol derivatives, extracts from plants viz. rubia and symplocos, hydroxycarboxylic acids like lactic acid and their salts e.g. sodium lactate, and mixtures thereof. Vitamin B3 compound or its derivative e.g. niacin, nicotinic acid or niacinamide are the more preferred skin lightening agent as per the invention, most preferred being niacinamide. Niacinamide, when used, is preferably 0.1 to 10 wt %, more preferably 0.2 to 5 wt %.

The compositions may further preferably comprise one or more organic sunscreens. A wide variety of organic sunscreen agents are suitable for use in combination with the essential ingredients of this invention. Suitable UV-A/UV-B sunscreen agents include, 2-hydroxy-4-methoxybenzophenone, octyldimethyl p-aminobenzoic acid, digalloyltrioleate, 2,2-dihydroxy-4-methoxybenzophenone, ethyl-4-(bis(hydroxypropyl)) aminobenzoate, 2-ethylhexyl-2-cyano-3,3-diphenylacrylate, 2-ethyl hexylsalicylate, glyceryl p-aminobenzoate, 3,3,5-trimethylcyclohexylsalicylate, methylanthranilate, p-dimethyl-aminobenzoic acid or aminobenzoate, 2-ethylhexyl-p-dimethyl-aminobenzoate, 2-phenylbenzimidazole-5-sulfonic acid, 2-(p-dimethylaminophenyl)-5-sulfonicbenzoxazoic acid, 2-ethylhexyl-p-methoxycinnamate, butylmethoxydibenzoylmethane, 2-hydroxy-4-methoxybenzophenone, octyldimethyl-p-aminobenzoic acid and mixtures thereof. The most suitable organic sunscreens are 2-ethylhexyl-p-methoxycinnamate, butylmethoxydibenzoylmethane or a mixture thereof.

A safe and effective amount of organic sunscreens is 0.1 to 10 wt %, more preferably from 0.1 to 5 wt %, of organic sunscreen agent.

Other materials which can be included in the cosmetically acceptable carrier include humectants, thickeners and powders. Examples of each of these types of material, which can be used singly or as mixtures, are as follows:

Humectants include those of the polyhydric alcohol-type. Typical polyhydric alcohols include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, glycerol, ethoxylated glycerol, propoxylated glycerol and mixtures thereof. The amount of humectant may range, for example, anywhere from 0.5 to 50%, more preferably between 1 and 15% by weight of the composition. Most preferred is glycerol (also known as glycerin). Amounts of glycerin may range, for example, from 0.5% to 50%, more preferably from 1 to 35%, optimally from 2 to 15% by weight of the composition.

A variety of thickening agents may be included in the compositions. Illustrative but not limiting are stearic acid, Acrylamide/Sodium Acryloyldimethyltaurate Copolymer (Aristoflex® AVC), Hydroxyethyl Acrylate/Sodium Acryloyldimethyltaurate Copolymer, Aluminum Starch Octenyl Succinate, Polyacrylates (such as Carbomers including Carbopol® 980, Carbopol® 1342, Pemulen TR-2® and the Ultrez® thickeners), Polysaccharides (including xanthan gum, guar gum, pectin, carageenan and sclerotium gums), celluloses (including carboxymethyl cellulose, ethyl cellulose, hydroxyethyl cellulose and methyl hydroxymethyl cellulose), minerals (including talc, silica, alumina, mica and clays, the latter being represented by bentonites, hectorites and attapulgites), magnesium aluminum silicate and mixtures thereof. Amounts of the thickeners may range, for example, from 0.05 to 10 wt %, more preferably from 0.3 to 2 wt % by weight of the composition.

Powders include chalk, talc, Fuller's earth, kaolin, starch, gums, colloidal silica sodium polyacrylate, tetraalkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose and ethylene glycol monostearate.

The composition can be formulated in any known format, more preferred formats being creams and lotions. The cosmetic compositions of the invention may further comprise other ingredients which are common in the art to enhance physical properties and performances. Suitable ingredients include but are not limited to binders, colorants and pigments, pH adjusting agents, preservatives, optics, perfumes, viscosity modifiers, biological additives, buffering agents, conditioners, natural extracts, essential oils and skin benefit agents including anti-inflammatory agents, cooling agents, antiperspirant agents, anti-aging agents, anti-acne agents, anti-microbial agents and antioxidants.

The cosmetic composition of this invention is a composition suitable for topical application to human skin, including leave-on and wash-off products. It is preferred that the compositions of the invention are leave-on compositions. It is more preferred that the cosmetic composition of this invention is a moisturizer, a vanishing cream or sun protection cream.

The compositions may be suitable packed in an appropriately sized packaging or dispenser. Packaging can be a jar or tube as well as any other format typically seen for cosmetic, cream, washing and lotion type products. The compositions may be applied topically and preferably 1 to 4 milligrams of composition is applied per square centimeter of skin.

### Method and Use

In a fourth aspect is disclosed a method of improving characteristics of skin comprising a step of topically applying thereon a cosmetic composition of the first aspect. Preferably the method is non-therapeutic in nature. Non-therapeutic nature means for cosmetic purposes. Alternatively, but less preferably, it is for therapeutic purpose.

In a fifth aspect is disclosed use of a composition of the first aspect for improving the characteristics of skin. The term improvement in characteristics of skin means to provide one or more of the benefits including natural looking skin, even=toned skin, translucent pinkish white skin or a combination thereof.

The following examples are provided to facilitate an understanding of the invention. The examples are not intended to limit the scope of the claims.

### EXAMPLES

Porous composite particles of this invention were prepared by the following process:

### Chemicals:

Ethyl acetate (Xilong Chemical Co., Ltd.); P(MMA-MAA), Poly(methyl methacrylate-co-methacrylic acid) (Sigma-Aldrich, MW 34000 D, MMA:MAA=65:1); Hydrophobic Fe2O3 (BRO-12®) nanoparticles (Kobo, size about 100 nm); sodium dodecyl sulfate, emulsifier (SDS) (Xilong Chemical Co., Ltd).

### Example 1

Preparation of small (particle size 5 μm) porous spherical composite particles with discontinuous outer surface comprising plurality of pores whose total area was 5% of the total outer surface area. The particles comprised base of PMMA (hydrophobic polymer) and iron oxide as the pigment.

(1) P(MMA-MAA) (16 g) was dissolved in Ethyl acetate (100 ml)-solvent in a 250 ml Erlenmeyer flask. And then Fe2O3 nanoparticles (0.35 g) was added to the solution and sonicated for 1 hour to make the particles well dispersed. The dispersion was centrifuged at 3000 rpm for 3 minutes to remove a few aggregates and the supernatant was used as oil phase. 0.1 wt % SDS solution (5.5 ml)-water phase was added to the oil phase (12.5 ml), and homogenized by IKA T18 (disperser/homogenizer Ex. Truelab®) at 20,000 rpm for 30 seconds to obtain a w/o emulsion.

(2) The w/o emulsion (18 ml) was added to 50 ml outer water phase with 0.05 g SDS and 0.05 g NaCl, and homogenized by IKA T18 at 11,200 rpm for 60 seconds to obtain a w/o/w emulsion.

(3) During stirring, the w/o/w emulsion was added to additional water (300 ml) in a 1000 ml beaker, and stirred at 400 rpm for 16 hours until the porous spherical composite particles were totally precipitated.

### Example 2

Preparation of large (particle size 20 μm) porous spherical composite particles with discontinuous outer surface comprising plurality of pores whose total area was 5% of the total outer surface area. The particles comprised base of PMMA (hydrophobic polymer) and iron oxide as the pigment.

The procedure is like Example 1, except the step 2 which is as follows:

The o/w emulsion (18 ml) was added to 50 ml outer water phase with 0.05 g SDS and 0.05 g NaCl, and homogenized by IKA T18 at 3,500 rpm for 60 seconds to obtain a w/o/w emulsion.

### Example 3

Preparation of large (particle size 20 μm) porous spherical composite particles with discontinuous outer surface comprising plurality of pores whose total area is 32% of the total outer surface area. The particles comprised base of PMMA (hydrophobic polymer) and iron oxide as the pigment. The particles of Example 3 were outside the scope of the invention.

The procedure is like Example 1, except steps 2 and 3 which are as follows.

### Step 2

The o/w emulsion (18 ml) was added to 50 ml outer water phase with 0.05 g SDS and 0.05 g NaCl, and homogenized by IKA T18 at 3,500 rpm for 60 seconds to obtain a w/o/w emulsion.

### Step 3

During stirring, the w/o/w emulsion was added to additional water (600 ml) in a 1000 ml beaker, and stirred at 400 rpm for 16 hours until the porous spherical composite particles were totally precipitate

### Example 4

This example demonstrates improvement of the sustainability factor of processing/making a cosmetic composition comprising porous composite particles of the invention when compared with conventional pigments outside the scope of the present invention. The example also demonstrates whitening and/or other color benefit of cosmetic compositions in accordance with the invention when compared with cosmetic compositions outside the scope of the present invention.

The various compositions that were prepared and tested are disclosed in Table 1. Some of the compositions were outside the scope of the present invention and reference thereto is also made in the Table.

TABLE 1

| | | Reference Code | | | | | |
|---|---|---|---|---|---|---|---|
| | | Wt % of the Ingredient | | | | | |
| Phase | Details of Ingredients | A | B | C | D | E | F |
| II | Porous composite | 0 | 2 | 0 | 0 | 0 | 0 |
| | particles (5 μm, | | | | | | |
| | 80 vol % internal | | | | | | |
| | porosity) | | | | | | |
| | Porous PMMA (5 μm) | 2 | 0 | 0 | 0 | 0 | 0 |
| | Fe₂O₃ | 0.04 | 0 | 0 | 0 | 0 | 0 |
| | Porous composite | 0 | 0 | 2 | 0 | 0 | 0 |
| | particles (5 μm, | | | | | | |
| | 71 vol % internal | | | | | | |
| | porosity) | | | | | | |
| | Porous composite | 0 | 0 | 0 | 2 | 0 | 0 |
| | particles (5 μm, | | | | | | |
| | 51 vol % internal | | | | | | |
| | porosity) | | | | | | |
| | Porous composite | 0 | 0 | 0 | 0 | 2 | 0 |
| | particle (20 μm, | | | | | | |
| | rough surface) | | | | | | |
| | Porous composite | 0 | 0 | 0 | 0 | 0 | 2 |
| | particles (20 μm, | | | | | | |
| | smooth surface) | | | | | | |
| I | Glycerin | 1 | 1 | 1 | 1 | 1 | 1 |
| | Carbomer | 0.4 | 0.4 | 0.4 | 0.4 | 0.4 | 0.4 |
| | Water | Balance to 100 wt | | | | | |
| | | % of the composition | | | | | |
| III | Isopropyl myristate | 2 | 2 | 2 | 2 | 1 | 2 |
| | Stearic Acid | 3 | 3 | 3 | 3 | 3 | 3 |
| | Cetyl Alcohol | 0.5 | 0.5 | 0.5 | 0.5 | 0.5 | 0.5 |
| | Mineral oil | 1.5 | 1.5 | 1.5 | 1.5 | 1.5 | 1.5 |
| | Glyceryl Stearate | 1.5 | 1.5 | 1.5 | 1.5 | 1.5 | 1.5 |
| | Dimethicone 200 cSt | 0.5 | 0.5 | 0.5 | 0.5 | 0.5 | 0.5 |
| IV | Neutralizing Agent | 1 | 1 | 1 | 1 | 1 | 1 |
| | (Base) | | | | | | |
| V | Niacinamide | 3 | 3 | 3 | 3 | 3 | 3 |
| VI | Preservative | 0.4 | 0.4 | 0.4 | 0.4 | 0.4 | 0.4 |

Note:
(i) Compositions B, C, D and F are within the invention; Composition A and E are outside the scope.
(ii) all Porous composite particles comprising Fe₂O₃ and PMMA comprised 2% Fe₂O₃;
(iii) smooth surface: means discontinuous outer surface comprising plurality of pores whose total area is 5% of the total outer surface area; and
(iv) rough surface: means discontinuous outer surface comprising plurality of pores whose total area is 32% of the total outer surface area.

Process for preparation of a cosmetic moisturizer cream:

The Phase I ingredients were put in a mixer of suitable size stirring till well dispersed. Thereafter, Phase II ingredients were added along with balance amount of the ingredients of Phase III, with homogenizer (for Composition A) or stirrer (Composition B-F) until well dispersion was complete. This was followed by addition of the ingredients of Phase IV. Finally, the Phase V and Phase ingredient VI were added to get cosmetic compositions in the form of creams.

### Test Methods:

### 1) Measurement of Whitening and Color Effect

For each sample of the cosmetic composition, a film was applied on a black draw-down card using a cubic film applicator of 75 microns. The L* and a* of the black card were measured before and after coating of sample by portable spectrophotometer CM2600d (MINOLTA Co. Ltd., Japan). ΔL*, Δa* stand for change in whiteness, redness after coating of sample compared to prior to coating respectively. A positive ΔL* indicates a whitening of the skin. A positive Δa* indicates a reddening of the skin.

### 2) Energy Consumption

Generally, when pigment (e.g. Fe2O3) nanoparticles are introduced into a moisturizer cream or vanishing cream, a homogenizer is needed at 4000 rpm (such as Silverson® L5M High Shear mixer, Power: 250 W) to ensure the nanoparticles are well dispersed in the cream. However, when porous composite particles are introduced into a moisturizer cream or vanishing cream, only a mechanical stirrer is required at 600 rpm (such as IKA RW20 mechanical stirrer, Power: 75 W) to ensure the porous composite particles are well dispersed in the cream.

Energy consumption=The power of the machine multiplied by time

The observations are tabulated in Tables 2 to 4.

TABLE 2

| | | | Energy consumption/ |
|---|---|---|---|
| Composition | Whitening and Color | | preparation of 1 kg |
| Reference number | ΔL* | Δa* | skin care cream |
| A of Table 1 | 13.27 | 4.41 | 0.125 kW · h |
| B of Table 1 | 14.58 | 4.04 | 0.0375 kW · h |

ΔL* value and Δa* observed in the case of composition A and B indicates that both co compositions can increase whiteness index (ΔL*) and redness index (Δa*), which is desirable for improving skin characteristics such as natural looking, even toned and translucent pinkish white skin. However, in terms of energy consumption to achieve such benefits, it is evident that the porous composite particles of the invention (composition B) could be mixed or dispersed uniformly in the carrier using significantly lesser energy which makes it clear that the process is more sustainable as compared to Composition A of Table 1 (out of the scope of present invention). For preparing 1 kg skin cream, 70% energy could be saved by including the porous composite particles in place of the conventional pigment. We believe that it is mainly because particle size of the porous composite particles is larger than primary nanoparticles. The aggregation of particles could be largely minimized to disperse with lesser energy.

TABLE 3
Effect of internal porosity

| Composition | | |
|---|---|---|
| Reference number | ΔL* | Δa* |
| B of Table 1 | 14.58 | 4.04 |
| C of Table 1 | 8.66 | 0.24 |
| D of Table 1 | 5.26 | 0.28 |

ΔL* value and Δa* observed in the case of composition B indicates that inclusion of the porous composite particles with high internal porosity (80 vol %) leads to increased whiteness index (ΔL*) and redness index (Δa*). On comparison with the data observed in the case of composition C (71 vol % internal porosity) and D (51 vol % internal porosity), it is evident that the increase in internal porosity of porous composite particles improves the whitening and/or other color effect which is desirable for improving skin characteristics such as natural looking, even toned and translucent pinkish white skin.

TABLE 4

| Composition | | |
|---|---|---|
| Reference number | ΔL* | Δa* |
| E of Table 1 | −0.1 | 0.16 |
| F of Table 1 | 3.88 | 0.412 |

ΔL* value and Δa* observed in the case of composition F indicates that inclusion of the porous composite particles with discontinuous outer surface comprising plurality of pores whose total area is 5% of the total outer surface area, leads to increased whiteness index (ΔL*) and redness index (Δa*). On comparison with the data observed in the case of composition E with discontinuous outer surface comprising plurality of pores whose total area is 32% of the total outer surface area, which is out of the scope of the invention, it is evident that when the porous composite particles having discontinuous outer surface as per the invention are included in a cosmetic composition, the total area of pores needs to be less than 30% of the total outer surface area to get desired whitening and/or other color effect.

1. Porous composite particles comprising base of an amphiphilic or hydrophobic polymer and 0.1 to 20% of a pigment based on the total weight of porous composite particles, where said particles have 50 to 95 vol % internal porosity and an outer surface which is either continuous and devoid of pores or is discontinuous comprising plurality of pores whose total area is 0.01 to 30% of the total outer surface area wherein said composite particles comprise a combination of pigments comprising a white pigment and non-white pigment;
wherein said porous composite particles are prepared by a process comprising the steps of:
(i) forming a w/o emulsion comprising a water phase and an oil phase, said oil phase comprising said polymer, an organic solvent for solubilizing the polymer and said pigment;
(ii) further emulsifying said w/o emulsion in an outer water phase comprising an emulsifier to form a w/o/w double emulsion;
(iii) mixing said w/o/w double emulsion with additional water to allow said solvent to diffuse into said additional water to precipitate said porous composite particles.

2. Porous composite particles as claimed in claim 1 wherein said total area is 0.1 to 10% of said total outer surface area.

3. Porous composite particles as claimed in claim 1 wherein said porous composite particles are spherical or spheroidal.

4. Porous composite particles as claimed in claim 3 wherein diameter of said porous composite particles is 0.5 to 100 microns.

5. Porous composite particles as claimed in claim 1 wherein said internal porosity is due to plurality of disconnected air pockets.

6. Porous composite particles as claimed in claim 1 wherein said internal porosity is due to plurality of interconnected air pockets.

7. Porous composite particles as claimed in claim 1 wherein particle size of said pigment is 0.01 to 5 microns.

8. Porous composite particles as claimed in claim 1 wherein said pigment is inorganic.

9. Porous composite particles as claimed in claim 8 wherein said pigment is iron oxide, titanium dioxide, zinc oxide, zirconium oxide or a mixture thereof.

10. (canceled)

11. Porous composite particles as claimed in claim 1 wherein said amphiphilic polymer is selected from methoxy polyethylene glycol polylactic acid copolymer, polyoxyethylene and polyoxypropylene copolymer, polystyrene and polyethylene glycol copolymer, polystyrene and polyacrylate copolymer, polystyrene and polyacrylate hydroxyethyl ester copolymer, polymethyl methacrylate and polyacrylate copolymer, polymethyl methacrylate and polyacrylate hydroxyethyl ester copolymer, methyl methacrylate and methacrylate copolymer, methyl methacrylate, β-hydroxyethyl methacrylate copolymer or mixture thereof.

12. Porous composite particles as claimed in claim 1 wherein said hydrophobic polymer is selected from polystyrene, polychloromethylstyrene, polyglycidyl methacrylate or a mixture thereof.

13. (canceled)

14. A cosmetic composition comprising porous composite particles as claimed in claim 1 in a cosmetically acceptable carrier.

15. A method of improving the characteristics of skin comprising a step of topically applying to skin the cosmetic composition as claimed in claim 14.
